(19) United States
(12) Patent Application Publication
(10) Pub. No.: US 2006/0147455 A1
(43) Pub. Date: Jul. 6, 2006

(54) Gastric and colon cancer-associated antigens

(75) Inventors: Robert Charles Rees, Sheffield (GB); Gene Li, Nottingham (GB); Aija Line, Riga (JP)

Correspondence Address: BANNER & WITCOFF, 1001 G STREET N W, SUITE 1100, WASHINGTON, DC, 20001, US

(73) Assignee: THE NOTTINGHAM TRENT UNIVERSITY, NOTTINGHAM (GB)

(21) Appl. No.: 10/523,981

(22) PCT Filed: Aug. 7, 2003
(86) PCT No.: PCT/GB03/03456

§ 371 Date: Feb. 8, 2005

(30) Foreign Application Priority Data
Aug. 8, 2002 (GB) 0218468.7

(51) Int. Cl. A61K 48/00; C12Q 1/68; G01N 33/574; A61K 39/395

(52) U.S. Cl. 424/155.1; 435/6; 435/7.23; 514/44

(57) ABSTRACT

The application discloses caner-associated genes and their products, especially those identifiable by SEREX. The genes and products are used to identify, track and treat cancer. Preferably the cancer is a gastro-intestinal cancer.

The invention relates to isolated nucleic acid sequences which are expressed in cancers, especially gastrointestinal cancers, to their protein products and to the use of the nucleic acid and protein products for the identification and treatment of cancers.

Cancers of the intestinal tract, such as gastric carcinomas and colorectal cancers, account for up to 15% of cancer-related deaths in the United States, and have low survival rates. Such cancers are often asymptomatic, the patient only becoming aware of them when the cancers have progressed too far to be successfully treated. There is therefore a need to identify new diagnostic tools and methods for treating such cancers.

Identification of immunogenic proteins in cancer is essential for the development of immunotherapeutic strategies where adoptive immunity is directed towards MHC Class I- and Class II-associated peptides (Mians, et al., Cancer Immunology (2001), page 1). Many antigens are implicated in aetiology and progression of cancer, and are associated with epigenetic events. Pre-clinical and clinical studies infer that vaccination and targeting MHC-associated peptide antigens promotes tumour rejection (Ali S. A., et al, J Immunol. (2002), Vol. 168(7), pages 3512-19 and Rees R. C., et al., Immunol. Immunother (2002), Vol 51(l), pages 58-61).

The inventors have used a technique known as SEREX (Serological Identification of Antigens by Recombinant Expression Cloning) to identify genes which are over-expressed in cancer tissue. This technique was published by Sahin et al (PNAS (USA), 1995, Vol. 92, pages 11810-11813). SEREX uses total RNA isolated from tumour biopsies from which poly(A)⁺ RNA is then isolated. cDNA is then produced using an oligo (dT) primer. The cDNA fragments produced are then cloned into a suitable expression vector, such as a bacteriophage and cloned into a suitable host, such as E. coli. The clones produced are screened with high-titer IgG antibodies in autologous patient serum, to identify antigens associated with the tumour.

Several SEREX-defined antigens have provided attractive candidates for the construction of cancer vaccines, for example NY-ESO-1 from testis (Chen Y. T., et al. (1997), Vol. ?4, page 1914; Stockert E., et al., J. Exp. Med. (1998), Vol. 187, page 1349; Jager E., et al. PNAS (2000), Vol. 97, page 12198; and Jager E., et al., PNAS (2000), Vol. 97, page 4760). Mutated p53 (Scanlan M. J., et al., Int. J. Cancer (1998), Vol. 76, page 652), putative tumour suppressor ING 1 (Jager D. et al., Cancer Res. (1999), Vol. 59, page 6416) and adhesion molecule galectin 9 (Tureci O., et al., J. Biol. Chem. (1997), Vol. 272, page 6416), for example, have been detected by SEREX, showing that the analysis of autoantibodies can identify genes involved in cancer etiology and identify diagnostic markers or indicators of disease progression.

The inventors have used this technique to identify genes and gene products associated with gastric cancer.

A first aspect of the invention provides an isolated mammalian nucleic acid molecule comprising a sequence selected from SEQ.ID.1 and SEQ.ID.2. Preferably the isolated nucleic acid molecule encodes a mammalian antigen which is expressed in higher than normal concentrations in cancer cells, compared with normal non-cancerous cells. Preferably the cancer is a gastro-intestinal cancer. The term “higher than normal concentrations” preferably means that the protein is expressed at a concentration at least 5 times greater in tumour cells than normal cells.

Preferably the nucleic acid molecule encodes TACC1-D (SEQ.ID.1).

TACC1 Splice Variant (TACC1-D); Full-Length mRNA

>acagccgcccgccgcccagcacaggagggtgcagccccggccccaagttctgcgccatgggaggctcccactctcagacccc
gaggggccgggaacccgccggggagaggcacccgagacccacggagaccgcgactactgaacaagtgaaatttctctgttttct
gttgagtggctgtaaggtgaagaagcatgaaactcagtctctcgccctggatgcatgttctcgggatgaaggggcagtgatctccca
gatttcagacatttctaatagggatggccatgctactgatgaggagaaactggcatccacgtcatgtggtcagaaatcagctggtgcc
gaggtgaaaggtgagccagaggaagacctggagtactttgaatgttccaatgttcctgtgtctaccataaatcatgcgttttcatcctca
gaagcaggcatagagaaggagacgtgccagaagatggaagaagacgggtccactgtgcttgggctgctggagtcctctgcagag
aaggcccctgtgtcggtgtcctgtggaggtgagagccccctggatgggatctgcctcagcgaatcagacaagacagccgtgctca
ccttaataagagaagagataattactaaagagattgaagcaaatgaatggaagaagaaatacgaagagacccggcaagaagttttg
gagatgaggaaaattgtagctgaatatgaaaagactattgctcaaatgattgaagatgaacaaaggacaagtatgacctctcagaag
agcttccagcaactgaccatggagaaggaacaggccctggctgaccttaactctgtggaaaggtccctttctgatctcttcaggagat
atgagaacctgaaaggtgttctggaagggttcaagaagaatgaagaagccttgaagaaatgtgctcaggattacttatccagagttaa
acaagaggagcagcgataccaggccctgaaaatccacgcagaagagaaactggacaaagccaatgaagagattgctcaggttcg
aacaaaagcaaaggctgagagtgcagctctccatgctggactccgcaaagagcagatgaaggtggagtccctggaaagggccct
gcagcagaagaaccaagaaattgaagaactgacaaaaatctgtgatgagctgattgcaaagctgggaaagactgactgagacact
ccccctgttagctcaacagatctgcatttggctgcttctcttgtgaccacaattatcttgccttatccaggaataattgcccctttgcagag
aaaaaaaaaaacttaaaaaaagcacatgcctactgctgcctgtcccgctttgctgccaatgcaacagccctggaagaaaccctagag
ggttgcatagtctagaaaggagtgtgacctgacagtgctggagcctcctagtttccccctatgaaggttcccttaggctgctgagtttg
ggtttgtgatttatctttagtttgttttaaagtcatctttactttcccaaatgtgttaaatttgtaactcctctttggggtcttctccaccacctgt
ctgatttttttgtgatctgtttaatcttttaattttttagtatcagtggttttatttaaggagacagtttggcctattgttacttccaatttataatca
agaaggggctctggatccccttttaaattacacacactctcacacacatacatgtatgtttatagatgctgctgctcttttccctgaagcat
agtcaagtaagaactgctctacagaaggacatatttccttggatgtgagaccctattttgaaatagagtcctgactcagaacaccaactt
aagaatttgggggattaaagatgtgaagaccacagtcttgggttttcatatctggagaagactatttgccatgacgttttgttgccctggt
atttggacactcctcagctttaatgggtgtggcccctttagggttagtcctcagactaatgatagtgtctgctttctgcatgaacggcaat
atgggactccctccaagctagggtttggcaagtctgccctagagtcatttactctcctctgcctccatttgttaatacagaatcaacattt
agtcttcattatctttttttttttttttgagacagagtttcgatctattttaagtatgtgaagaaaatctacttgtaaaaggctcagatcttaatta
aaaggtaattgtagcacattaccaattataaggtgaagaaatgtttttttcccaagtgtgatgcattgttcttcagatgttgaaaagaaagc
aaaaaataccttctaacttaagacagaatttttaacaaaatgagcagtaaaagtcacatgaaccactccaaaaatcagtgcattttgcat
atttttaaacaaagacagcttgttgaatactgagaagaggagtgcaaggagaaggtctgtactaacaaagccaaattcctcaagctctt
actggactcagttcagagtggtgggccattaaccccaacatggaatttttccatataaatctcaatgaattccctttcatttgaataggca
aacccaaatccatgcaagtgttttaaagcactgtcctgtcttaatcttacatgctgaaagtcttcatggtgatatgcactatattcagtata
cgtatgttttcctacttctcttgtaaaactgttgcatgatccaacttcagcaatgaattgtgcctagtggagaacctctatagatcttaaaaa
atgaattattctttagcagtgtattactcacatgggtgcaatctttagccccagggaggtcaataatgtcttttaaagccagaagtcacatt
ttaccaatatgcatttatcataattggtgcttaggctgtatattcaagcctgttgtcttaacattttgtataaaaaagaacaacagaaattatc
tgtcatttgagaagtggcttgacaatcatttgagctttgaaagcagtcactgtggtgtaatatgaatgctgtcctagtggtcatagtacca
agggcacgtgtctccccttggtataactgatttcctttttagtcctctactgctaaataagttaattttgcattttgcagaaagaaacattgat
tgctaaatctttttgctgctgtgttttggtgttttcatgtttacttgttttatattgatctgttttaagtatgagaggcttatagtgccctccattgt
aaatccatagtcatctttttaagcttattgtgtttaagaaagtagctatgtgttaaacagaggtgatggcagcccttccctagcacactggt
ggaagagaccccttaagaacctgaccccagtgaatgaagctgatgcacagggagcaccaaaggaccttcgttaagtgataattgtc
ctggcctctcagccatgaccgttatgaggaaatatcccccattcgaacttaacagatgcctcctctccaaagagaattaaaatcgtagc
ttgtacagatcaagagaatatactgggcagaatgaagtatgtttgtttatttttctttaaaaataaaggattttggaactctggagagtaag
aatatagtatagagtttgcctcaacacatgtgagggccaaataacctgctagctaggcagtaataaactctgttacagaagagaaaaa
gggccgggcacagtggcttattcctgtaatcccaacactgtggaaggccgaggcaggaggatcacttgagtccaggagtttgaaac
ctacctaggcaacatggtgaaaccttgtctctaccaaaataaaaattagctgggcatggtggcacgtgcctgtggtcccagctacttg
ggaggctgaggtgggagcctgggaggtcaaggctgcagtgagccatgatcatgccactgcactccatcctgggtgacagcaagat
cttgtctcaaaaaaaaaaaaaaaaaaaaaaaacccaggagtgaaaaaggaaagtaaaaggcagctgctggcctaaatgttggtttg
ggaatattaggtgatcctgttgaaattctggatccaaagcaatttctttagcttttgactttgccaaagtgtaaatagcctttatccaccagt
tttttaaggggggaatgcaacgggaggccaactgaacaattccccccgtggctgcccagatagtcacagtcaaggttggagagtct
ccttccagccagggacctacccaaaccttttgttctgtaaaactgctctggaaataccgggaagcccagttttctcacgtggtttctagc
ttcttcggactcagcccaatttaggagtgccgaagcacatgatgg//

Transforming acidic coiled-coil (TACC) proteins are centrosome and microtubule-associated proteins that are essential for mitotic spindle production (Gergely F., et al., PNAS (USA) (2000), Vol. 97, pages 14352-57; Gergely F. et al., EMBO J. (2000), Vol. 19, pages 241-252; and Lee M. J., et al., Nat. Cell. Biol., Vol. 3, pages 543-649). TACC-1 in mouse fibroblasts, when over-expressed, results in cellular transformation and anchorage independent growth (Still I. H., et al., Oncogene (1999), Vol. 18, pages 4032-4038). High levels of TACC-3 mRNA have been found in various cancer cell lines (Still I. H., Genomics (1999), Vol. 58, pages 165-170) but TACC-2 (AZU-1) has been identified as a potential breast tumour suppressor and is downregulated in breast carcinoma cell lines (Chen H. M., Mol. Biol. Cell (2000), Vol. 11, pages 1357-1367).

TACC-1 has now been identified as an immunogenic protein and a potential tumour antigen. 5′RLM-RACE and RT-PCR analysis identified a transcript variant, designated TACC1-D as being relatively strongly expressed in 50% of gastric tissue samples analysed. The variant is only weakly detectable in normal kidney and colon tissues but not in other normal tissues.

Five other TACC-1 splice variants have also been found (TACC1-A, TACC1-B, TACC1-C, TACC1-E and TACC1-F). TACC1-A, TACC1-B, TACC1-C and TACC1-E were expressed universally in all normal tissues tested. TACC1-F was expressed in brain and gastric tumous to a similar level.

Preferably the isolated nucleic acid sequence encodes AD034 (SEQ.ID.2).

AD 034 mRNA Sequence

| | |
|---|---|
| 1 | gggtggtgga tctgtcggtc ccgttttccc gtcgcacgtg |
| | gtggccactg ttggcttctg |
| 61 | aatggtttgc aaggcggata tccacgccaa ggcctttgga |
| | tcggccgtgg gtacatccgt |
| 121 | ctgagccgtt cctttccatc gcagagcggc ggcctccggc |
| | ggcgctctcc agtcatggac |
| 181 | taccggcggc ttctcatgag ccgggtggtc cccgggcaat |
| | tcgacgacgc ggactcctct |
| 241 | gacagtgaaa acagagactt gaagacagtc aaagagaagg |
| | atgacattct gtttgaagac |
| 301 | cttcaagaca atgtgaatga gaatggtgaa ggtgaaatag |
| | aagatgagga ggaggagggt |
| 361 | tatgatgatg atgatgatga ctgggactgg gatgaaggag |
| | ttggaaaact cgccaagggt |
| 421 | tatgtctgga atggaggaag caacccacag gcaaatcgac |
| | agacctccga cagcagttca |
| 481 | gccaaaatgt ctactccagc agacaaggtc ttacggaaat |
| | ttgagaataa aattaattta |
| 541 | gataagctaa atgttactga ttccgtcata aataaagtca |
| | ccgaaaagtc tagacaaaag |
| 601 | gaagcagata tgtatcgcat caaagataag gcagacagag |
| | caactgtaga acaggtgttg |
| 661 | gatcccagaa caagaatgat tttattcaag atgttgacta |
| | gaggaatcat aacagagata |
| 721 | aatggctgca ttagcacagg aaaagaagct aatgtatacc |
| | atgctagcac agcaaatgga |
| 781 | gagagcagag caatcaaaat ttataaaact tctattttgg |
| | tgttcaaaga tcgggataaa |
| 841 | tatgtaagtg gagaattcag atttcgtcat ggctattgta |
| | aaggaaaccc taggaaaatg |
| 901 | gtgaaaactt gggcagaaaa agaaatgagg aacttaatca |
| | ggctaaacac agcagagata |
| 961 | ccatgtccag aaccaataat gctaagaagt catgttcttg |
| | tcatgagttt catcggtaaa |
| 1021 | gatgacatgc ctgcaccact cttgaaaaat gtccagttat |
| | cagaatccaa ggctcgggag |
| 1081 | ttgtacctgc aggtcattca gtacatgaga agaatgtatc |
| | aggatgccag acttgtccat |
| 1141 | gcagatctca gtgaatttaa catgctgtac cacggtggag |
| | gcgtgtatat cattgacgtg |
| 1201 | tctcagtccg tggagcacga ccacccacat gccttggagt |
| | tcttgagaaa ggattgcgcc |
| 1261 | aacgtcaatg atttctttat gaggcacagt gttgctgtca |
| | tgactgtgcg ggagctcttt |
| 1321 | gaatttgtca cagatccatc cattacacat gagaacatgg |
| | atgcttatct ctcaaaggcc |
| 1381 | atggaaatag catctcaaag gaccaaggaa gaacggtcta |
| | gccaagatca tgtggatgaa |
| 1441 | gaggtgttta agcgagcata tattcctaga accttgaatg |
| | aagtgaaaaa ttatgagagg |
| 1501 | gatatggaca taattatgaa attgaaggaa gaggacatgg |
| | ccatgaatgc ccaacaagat |
| 1561 | aatattctat accagactgt tacaggattg aagaaagatt |
| | tgtcaggagt tcagaaggtc |
| 1621 | cctgcactcc tagaaaatca agtggaggaa aggacttgtt |
| | ctgattcaga agatattgga |
| 1681 | agctctgagt gctctgacac agactctgaa gagcagggag |
| | accatgcccg ccccaagaaa |
| 1741 | cacaccacgg accctgacat tgataaaaaa gaaagaaaaa |
| | agatggtcaa ggaagcccag |
| 1801 | agagagaaaa gaaaaaacaa aattcctaaa catgtgaaaa |
| | aaagaaagga gaagacagcc |
| 1861 | aagacgaaaa aaggcaaata gaatgagaac catattatgt |
| | acagtcattt tcctcagttc |
| 1921 | cttttctcgc ctgaactctt aagctgcatc tggaagatgg |
| | cttattggtt ttaaccagat |
| 1981 | tgtcatcgtg gcactgtctg tgaagacgga ttcaaatgtt |
| | ttcatgtaac tatgtaaaaa |
| 2041 | gctctaagct ctagagtcta gatccagtca ctgactctgt |
| | ctggtgttga cagaggattt |
| 2101 | atttaagcta ttattttaat aaagaacttt gtacattttt |
| | atttttatat ttttttctct |
| 2161 | tacaaatatg tttttggaag catgataaat gtttaaatgt |
| | agtcaacatc tgtaactctt |
| 2221 | acatgagtgt ccagaggcac tcatgggaaa attggttttg |
| | ctttctttgt acacaccaga |
| 2281 | gacccatctg aggtcatctg attataaggc catgtttata |
| | taaagggaat ttcacccaca |
| 2341 | gttcagctgg ctgttgattt tcactgcaac tctgcctttg |
| | tgtgtattgg cgatcatttg |
| 2401 | taatgctctt acacttcgtc tttaatgttc tttttggagt |
| | taggacctct cagttcataa |
| 2461 | agttttttac aattcaaaaa aaaaaaaaaa aaaaa |

AD034 encodes a tyrosine kinase motif and has similarity to the RIO1/ZK632.3/MJ0444 family. RT-PCR showed that the protein contains a 32-bp frame shift mutation which is not associated with the increased levels observed in colorectal cancer patients. The 32-bp sequence is a minor mRNA variant and is detectable in normal tissues where AD034 is expressed and no significant differences in ratios of either isoform were observed between colorectal tumours and adjacent normal tissues.

cDNA Sequence of AD034 with 32bp Insertion (SEQ.ID 3)

| | |
|---|---|
| 1 | gggtggtgga tctgtcggtc ccgttttccc gtcgcacgtg gtggccactg ttqgcttctg |
| 61 | aatggtttgc aaggcggata tccacgccaa ggcctttgga tcggccgtgg gtacatccgt |
| 121 | ctgagccgtt cctttccatc gcagagcggc ggcctccggc ggcgctctcc agtcatggac |
| 181 | taccggcggc ttctcatgag ccgggtggtc cccgggcaat tcgacgacgc ggactcctct |
| 241 | gacagtgaaa acagagactt gaagacagtc aaagagaagg atgacattct gtttgaagac |
| 302 | cttcaagaca atgtgaatga gaatggtgaa ggtgaaatag aagatgagga ggaggagggt |
| 361 | tatgatgatg atgatgatga ctqggactgg gatgaaggag ttggaaaact cgccaagggt |
| 422 | tatgtctgga atggaggaag caacccacagCTAGTGCCTTAGACTCTGGAATTCCCTTCTAG |
| | gcaaatcgac agacctccga cagcagttca |
| 481 | gccaaaatgt ctactccagc agacaaggtc ttacggaaat ttgagaataa aattaattta |
| 541 | gataagctaa atgttactga ttccgtcata aataaagtca ccgaaaagtc tagacaaaag |
| 601 | gaagcagata tgtatcgcat caaagataag gcagacagag caactgtaga acaggtgttg |
| 661 | gatcccagaa caagaatgat tttattcaag atgttgacta gaggaatcat aacagagata |
| 721 | aatggctgca ttagcacagg aaaagaagct aatgtatacc atgctagcac agcaaatgga |
| 781 | gagagcagag caatcaaaat ttataaaact tctattttgg tgttcaaaga tcgggataaa |
| 841 | tatgtaagtg gagaattcag atttcgtcat ggctattgta aaggaaaccc taggaaaatg |
| 901 | gtgaaaactt gggcagaaaa agaaatgagg aacttaatca ggctaaacac agcagagata |
| 961 | ccatgtccag aaccaataat gctaagaagt catgttcttg tcatgagttt catcggtaaa |
| 1021 | gatgacatgc ctgcaccact cttgaaaaat gtccagttat cagaatccaa ggctcgggag |
| 1081 | ttgtacctgc aggtcattca gtacatgaga agaatgtatc aggatgccag acttgtccat |
| 1141 | gcagatctca gtgaatttaa catgctgtac cacggtggag gcgtgtatat cattgacgtg |
| 1201 | tctcagtccg tggagcacga ccacccacat gccttggagt tcttgagaaa ggattgcgcc |
| 1261 | aacgtcaatg atttctttat gaggcacagt gttgctgtca tgactgtgcg ggagctcttt |
| 1321 | gaatttgtca cagatccatc cattacacat gagaacatgg atgcttatct ctcaaaggcc |
| 1381 | atggaaatag catctcaaag gaccaaggaa gaacggtcta gccaagatca tgtggatgaa |
| 1441 | gaggtgttta agcgagcata tattcctaga accttgaatg aagtgaaaaa ttatgagagg |
| 1501 | gatatggaca taattatgaa attgaaggaa gaggacatgg ccatgaatgc ccaacaagat |
| 1561 | aatattctat accagactgt tacaggattg aagaaagatt tgtcaggagt tcagaaggtc |
| 1621 | cctgcactcc tagaaaatca agtggaggaa aggacttgtt ctgattcaga agatattgga |
| 1681 | agctctgagt gctctgacac agactctgaa gagcagggag accatgcccg ccccaagaaa |
| 1741 | cacaccacgg accctgacat tgataaaaaa gaaagaaaaa agatggtcaa ggaagcccag |
| 1801 | agagagaaaa gaaaaaacaa aattcctaaa catgtgaaaa aaagaaagga gaagacagcc |
| 1861 | aagacgaaaa aaggcaaata gaatgagaac catattatgt acagtcattt tcctcagttc |
| 1921 | cttttctcgc ctgaactctt aagctgcatc tggaagatgg cttattggtt ttaaccagat |
| 1981 | tgtcatcgtg gcactgtctg tgaagacgga ttcaaatgtt ttcatgtaac tatgtaaaaa |
| 2041 | gctctaagct ctagagtcta gatccagtca ctgactctgt ctggtgttga cagaggattt |
| 2101 | atttaagcta ttattttaat aaagaacttt gtacattttt atttttatat ttttttctct |
| 2161 | tacaaatatg tttttggaag catgataaat gtttaaatgt agtcaacatc tgtaactctt |
| 2221 | acatgagtgt ccagaggcac tcatgggaaa attggttttg ctttctttgt acacaccaga |
| 2281 | gacccatctg aggtcatctg attataaggc catgtttata taaagggaat ttcacccaca |
| 2341 | gttcagctgg ctgttgattt tcactgcaac tctgcctttg tgtgtattgg cgatcatttg |
| 2401 | taatgctctt acacttcgtc tttaatgttc tttttggagt taggacctct cagttcataa |
| 2461 | agttttttac aattcaaaaa aaaaaaaaaa aaaaa |

The insertion is shown in upper case letters.

Fragments of the nucleic acid molecules which encode antigenic determinants unique to each protein are also included.

Preferably such determinants are specific for TACC1-D and do not cross-react with, e.g. TACC1-A, TACC1-B, TACC1-C, TACC1-E or TACC1-F.

Preferably the determinants are specific for AD034 with or without its insertion.

Nucleic acid molecules having at least 60%, 70%, 75%, 80%, 85%, 90%, 95%, 98%, 99% homology to the nucleic acid molecules are also provided. Preferably these have TACC1-D activity or AD034 activity.

The invention also includes, within its scope, nucleic acid molecules complementary to such isolated mammalian nucleic acid molecules.

The nucleic acid molecules of the invention may be DNA, cDNA or RNA. In RNA molecules “T” (Thymine) residues may be replaced by “U” (Uridine) residues.

Preferably, the isolated mammalian nucleic acid molecule is an isolated human nucleic acid molecule.

The invention further provides nucleic acid molecules comprising at least 15 nucleotides capable of specifically hybridising to a sequence included within the sequence of a nucleic acid molecule according to the first aspect of the invention. The hybridising nucleic acid molecule may either be DNA or RNA. Preferably the molecule is at least 90%, at least 92%, at least 94%, at least 96%, at least 98%, at least 99%, homologous to the nucleic acid molecule according to the first aspect of the invention. This may be determined by techniques known in the art.

The term “specifically hybridising” is intended to mean that the nucleic acid molecule can hybridise to nucleic acid molecules according to the invention under conditions of high stringency. Typical conditions for high stringency include 0.1×SET, 0.1% SDS at 68° C. for 20 minutes.

The invention also encompasses variant DNAs and cDNAs which differ from the sequences identified above, but encode the same amino acid sequences as the isolated mammalian nucleic acid molecules, by virtue of redundancy in the genetic code.

| | U | C | A | G | |
|---|---|---|---|---|---|
| U | 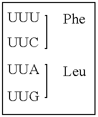 | 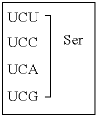 | 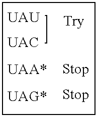 | 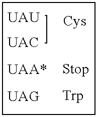 | 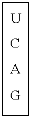 |
| C | 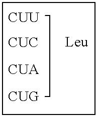 | 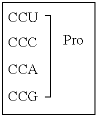 | 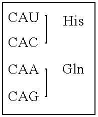 | 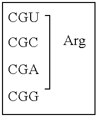 | 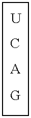 |
| A | 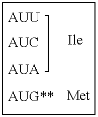 | 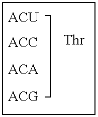 | 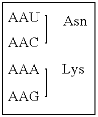 | 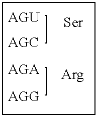 | 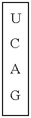 |
| G | 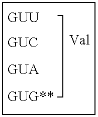 | 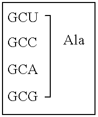 | 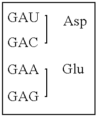 | 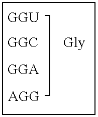 | 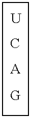 |

*Chain-terminating, or “nonsense” codons.
**Also used to specify the initiator formyl-Met-tRNAMet. The Val triplet GUG is therefore “ambiguous” in that it codes both valine and methionine.

The genetic code showing mRNA triplets and the amino acids for which they code

The invention also includes within its scope vectors comprising a nucleic acid according to the invention. Such vectors include bacteriophages, phagemids, cosmids and plasmids. Preferably the vectors comprise suitable regulatory sequences, such as promoters and termination sequences which enable the nucleic acid to be expressed upon insertion into a suitable host. Accordingly, the invention also includes hosts comprising such a vector. Preferably the host is E.coli.

A second aspect of the invention provides an isolated polypeptide obtainable from a nucleic acid sequence according to the invention. As indicated above, the genetic code for translating a nucleic acid sequence into an amino acid sequence is well known.

Preferably the sequence is:

AD034 Peptide Sequence

/translation=“MSRVVPGQFDDADSSDSENRDLKTVKEKDDILFEDL
QDNVNENGEGEIEDEEEEGYDDDDDDWDWDEGVGKLAKGYVWNGGSNPQA
NRQTSDSSSAKMSTPADKVLRKFENKINLDKLNVTDSVINKVTEKSRQKE
ADMYRIKDKADRATVEQVLDPRTRMILFKMLTRGIITEINGCISTGKEAN
VYHASTANGESRAIKIYKTSILVFKDRDKYVSGEFRFRHGYCKGNPRKMV
KTWAEKEMRNLIRLNTAEIPCPEPIMLRSHVLVMSFIGKDDMPAPLLKNV
QLSESKARELYLQVIQYMRRMYQDARLVHADLSEFNMLYHGGGVYIIDVS
QSVEHDHPHALEFLRKDCANVNDFFMRHSVAVMTVRELFEFVTDPSITHE
NMDAYLSKAMIEASQRTKEERSSQDHVDEEVFKRAYIPRTLNEVKNYERD
MDIIMKLKEEDMAMNAQQDNILYQTVTGLKKDLSGVQKVPALLENQVEER
TCSDSEDIGSSECSDTDSEEQGDHARPKKHTTDPDIDKKERKKMVKEAQR
EKRKNKIPKHVKKRKEKTAKTKKGK

The invention further provides polypeptide analogues, fragments or derivatives of antigenic polypeptides which differ from naturally-occurring forms in terms of the identity of location of one or more amino acid residues (deletion analogues containing less than all of the residues specified for the protein, substitution analogues wherein one or more residues specified are replaced by other residues in addition analogues wherein one or more amino acid residues are added to a terminal or medial portion of the polypeptides) and which share some or all properties of the naturally-occurring forms. Preferably such polypeptides comprise between 1 and 20, preferably 1 and 10 amino acid deletions or substitutions.

Preferably the polypeptide is at least 95%, 96%, 97%, 98% or 99% identical to the sequences of the invention. This can be determined conventionally using known computer programs such as the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

The nucleic acids and polypeptide of the invention are preferably identifiable using the SEREX method. However, alternative methods, known in the art, may be used to identify nucleic acids and polypeptides of the invention. These include differential display PCR (DD-PCR), representational difference analysis (RDA) and suppression subtracted hybridisation (SSH).

All of the nucleic acid molecules according to the invention and the polypeptides which they encode are detectable by SEREX (discussed below). The technique uses serum antibodies from cancer patients to identify the molecules. It is therefore the case that the gene products identified by SEREX are able to evoke an immune response in a patient and may be considered as antigens suitable for potentiating further immune reactivity if used as a vaccine.

The third aspect of the invention provides the use of nucleic acids or polypeptides according to the invention, to detect or monitor cancers, preferably gastrointestinal cancers, such as gastric cancer or colorectal cancer.

The use of a nucleic acid molecule hybridisable under high stringency conditions, a nucleic acid according to the first aspect of the invention to detect or monitor cancers, e.g. gastro-intestinal cancers, such as gastric cancer or colorectal cancer, is also encompassed. Such molecules may be used as probes, e.g. using PCR.

The expression of genes, and detection of their polypeptide products may be used to monitor disease progression during therapy or as a prognostic indicator of the initial disease status of the patient There are a number of techniques which may be used to detect the presence of a gene, including the use of Northern blot and reverse transcription polymerase chain reaction (RT-PCR) which may be used on tissue or whole blood samples to detect the presence of cancer associated genes. For polypeptide sequences in-situ staining techniques or enzyme linked ELISA assays or radio-immune assays may be used. RT-PCR based techniques would result in the amplification of messenger RNA of the gene of interest (Sambrook, Fritsch and Maniatis, Molecular Cloning, A Laboratory Manual, 2^{nd} Edition). ELISA based assays necessitate the use of antibodies raised against the protein or peptide sequence and may be used for the detection of antigen in tissue or serum samples (McIntyre C. A., Rees R. C. et. al., Europ. J. Cancer 28, 58-631 (1990)). In-situ detection of antigen in tissue sections also rely on the use of antibodies, for example, immuno peroxidase staining or alkaline phosphatase staining (Gaepel, J. R., Rees, R. C. et.al., Brit. J. Cancer 64, 880-883 (1991)) to demonstrate expression. Similarly radio-immune assays may be developed whereby antibody conjugated to a radioactive isotope such as I¹²⁵ is used to detect antigen in the blood.

Blood or tissue samples may be assayed for eleviated concentrations of the nucleic acid molecules or polypeptides.

Methods of producing antibodies which are specific to the polypeptides of the invention, for example, by the method of Kohler & Milstein to produce monoclonal antibodies, are well known. A further aspect of the invention provides an antibody which specifically binds to a polypeptide according to the invention.

Preferably, for example, the antibody binds TACC1-D, and not TACC1-A, TACC1-B, TACC1-C, TACC1-E or TACC1-F.

Kits for detecting or monitoring cancer, such as gastro-intestinal cancers, including gastric cancer and/or colorectal cancer, using polypeptides, nucleic acids or antibodies according to the invention are also provided. Such kits may additionally contain instructions and reagents to carry out the detection or monitoring.

The fourth aspect of the invention provides for the use of nucleic acid molecules according to the first aspect of the invention or polypeptide molecules according to the second aspect of the invention in the prophylaxis or treatment of cancer, or pharmaceutically effective fragments thereof. By pharmaceutically effective fragment, the inventors mean a fragment of the molecule which still retains the ability to be a prophylactant or to treat cancer. The cancer may be a gastro-intestinal cancer, such as gastric cancer or colorectal cancer.

The molecules are preferably administered in a pharmaceutically amount. Preferably the dose is between 1 μg/kg. to 10 mg/kg.

The nucleic acid molecules may be used to form DNA-based vaccines. From the published literature it is apparent that the development of protein, peptide and DNA based vaccines can promote anti-tumour immune responses. In pre-clinical studies, such vaccines effectively induce a delayed type hypersensitivity response (DTH), cytotoxic T-lymphocyte activity (CTL) effective in causing the destruction (death by lysis or apoptosis) of the cancer cell and the induction of protective or therapeutic immunity. In clinical trials peptide-based vaccines have been shown to promote these immune responses in patients and in some instances cause the regression of secondary malignant disease. Antigens expressed in prostate cancer (or other types of cancers) but not in normal tissue (or only weakly expressed in normal tissue compared to cancer tissue) will allow us to assess their efficacy in the treatment of cancer by immunotherapy. Polypeptides derived from the tumour antigen may be administered with or without immunological adjuvant to promote T-cell responses and induce prophylactic and therapeutic immunity. DNA-based vaccines preferably consist of part or all of the genetic sequence of the tumour antigen inserted into an appropriate expression vector which when injected (for example via the intramuscular, subcutaneous or intradermal route) cause the production of protein and subsequently activate the immune system. An alternative approach to therapy is to use antigen presenting cells (for example, dendritic cells, DC's) either mixed with or pulsed with protein or peptides from the tumour antigen, or transfect DC's with the expression plasmid (preferably inserted into a viral vector which would infect cells and deliver the gene into the cell) allowing the expression of protein and the presentation of appropriate peptide sequences to T-lymphocytes.

Accordingly, the invention provides a nucleic acid molecule according to the invention in combination with a pharmaceutically-acceptable carrier.

A further aspect of the invention provides a method of prophylaxis or treatment of a cancer such as a gastro-intestinal cancer comprising the administration to a patient of a nucleic acid molecule according to the invention.

The polypeptide molecules according to the invention may be used to produce vaccines to vaccinate against a cancer, such as a gastro-intestinal cancer.

Accordingly, the invention provides a polypeptide according to the invention in combination with a pharmaceutically acceptable carrier.

The invention further provides use of a polypeptide according to the invention in a prophylaxis or treatment of a cancer such as a gastro-intestinal cancer.

Methods of prophylaxis or treating a cancer, such as a gastro-intestinal cancer, by administering a protein or peptide according to the invention to a patient, are also provided.

Vaccines comprising nucleic acid and/or polypeptides according to the invention are also provided.

The polypeptides of the invention may be used to raise antibodies. In order to produce antibodies to tumour-associated antigens procedures may be used to produce polyclonal antiserum (by injecting protein or peptide material into a suitable host) or monoclonal antibodies (raised using hybridoma technology). In addition PHAGE display antibodies may be produced, this offers an alternative procedure to conventional hybridoma methodology. Having raised antibodies which may be of value in detecting tumour antigen in tissues or cells isolated from tissue or blood, their usefulness as therapeutic reagents could be assessed. Antibodies identified for their specific reactivity with tumour antigen may be conjugated either to drugs or to radioisotopes. Upon injection it is anticipated that these antibodies localise at the site of tumour and promote the death of tumour cells through the release of drugs or the conversion of pro-drug to an active metabolite. Alternatively a lethal effect may be delivered by the use of antibodies conjugated to radioisotopes. In the detection of secondary/residual disease, antibody tagged with radioisotope could be used, allowing tumour to be localised and monitored during the course of therapy.

The term “antibody” includes intact molecules as well as fragments such as Fa, F(ab′)₂ and Fv.

The invention accordingly provides a method of treating a gastrointestinal cancer by the use of one or more antibodies raised against a polypeptide of the invention.

The cancer-associated proteins identified may form targets for therapy.

The invention also provides nucleic acid probes capable of binding sequences of the invention under high stringency conditions. These may have sequences complementary to the sequences of the invention and may be used to detect mutations identified by the inventors. Such probes may be labelled by techniques known in the art, e.g. with radioactive or fluorescent labels.

Preferably the gastro-intestinal cancer which is detected, assayed for, monitored, treated or targeted for prophylaxis, is a gastric cancer or a colorectal cancer. Most preferably, the cancer is a gastric carcinoma or a colonic carcinoma, more preferably a gastric adenocarcinoma or a colonic adenocarcinoma.

The invention will now be described by reference to the following figure and examples:

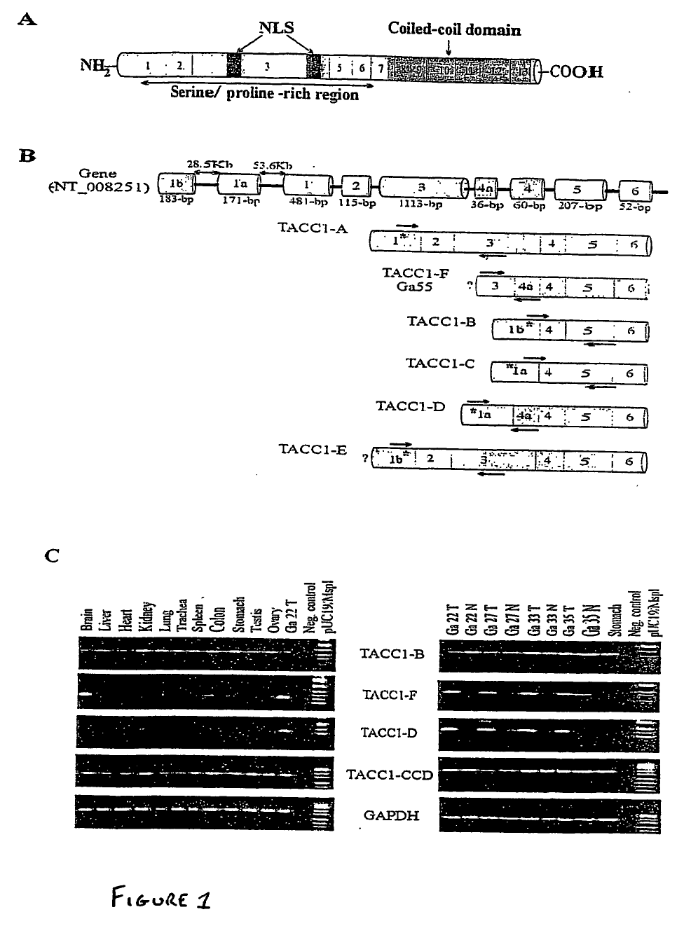

FIG. 1

(A) Schematic representation of TACC1-A exon composition and functional domains of the protein. Putative coiled-coil domain and nuclear localization signals (NLS) were predicted using sequence analysis tools at the SEREX web-site (http://www-ludwig.unil.cb/SEREX)

(B) The 5′ region of TACC1 gene and the mRNA variants identified by 5′RLM-RACE. Exon-intron composition of TACC1 was determined by comparing the cDNA sequences with the working draft of the human genome. The complete 5′ end sequences of TACC1-F and -E variants are not known. Potential translation initiation codons are marked with an asterisk but primers for expression analysis are indicated by arrows.

(C) Expression of the identified TACC1 mRNA variants in normal tissues and 4 specimens of gastric cancer (T) and adjacent normal tissues (N) analysed by RT-PCR. Amplification of GAPDH and TACC-CCD (coiled-coil domain, exons 8-11) was determined to be within the linear phase thus allowing comparison of mRNA levels.

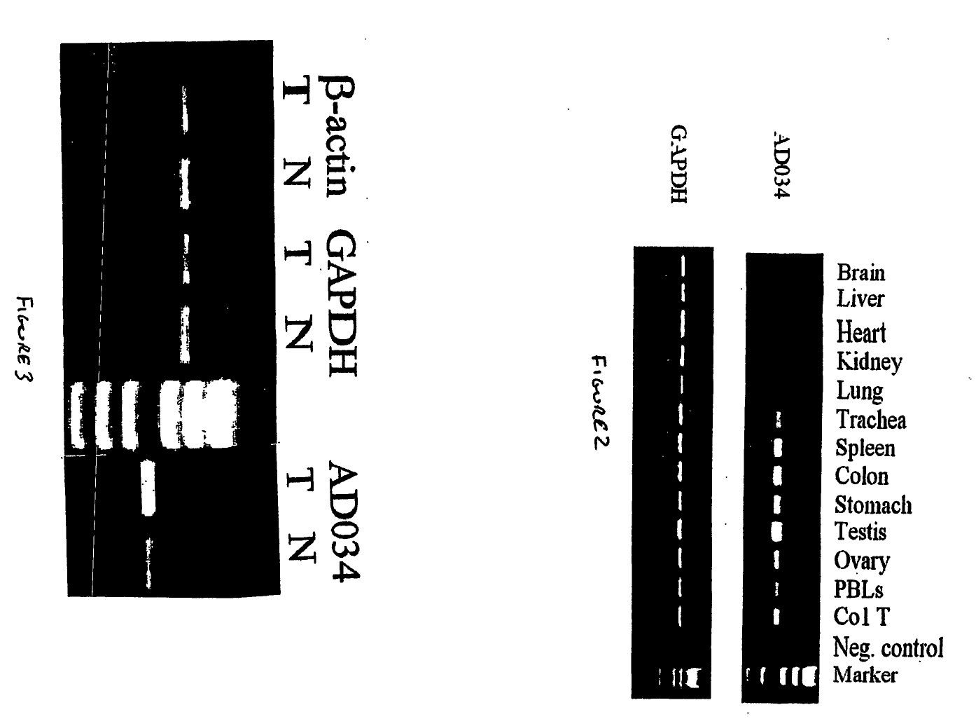

FIG. 2. Expression of AD034 mRNA in normal tissues and autologous tumour (Col T) analysed by RT-PCR. GAPDH was amplified as an internal control and demonstrates the equal amounts of mRNA used for RT-PCR.

FIG. 3. An example of comparison of AD034 mRNA levels between cancerous and adjacent non-cancerous tissues by RT-PCR. Cycling conditions were optimised so that the RT-PCR products were analysed when amplification is within the linear phase. Ethidium bromide stained gels were scanned on digital gel documentation system, intensities of bands were calculated and relative expression coefficients were determined using standard curves of amplification and expression of each target gene was normalised to that of β-actin and GAPDH. In the example showed, 4.8-fold increase (the mean values of two independent experiments) of AD034 in cancerous tissues was observed (normalised to β-actin).

### TECHNIQUE USED TO IDENTIFY GENES ENCODING TUMOUR ANTIGENS (SEREX TECHNIQUE)

The technique for the expression of cDNA libraries from human tissue moderately differentiated, ulcerated gastric adenocarcinoma and moderately differentiated colon adenocarcinoma is described, and was performed according to published methodology (Sahin et.al. Proc Natl. Acad. Sci. 92, 11810-11813, 1995).

SEREX has been used to analyze gene expression in tumour tissues from human melanoma, renal cell cancer, astrocytoma, oesophageal squamous cell carcinoma, colon cancer, lung cancer and Hodgkin's disease. Sequence analysis revealed that several different antigens, including HOM-MEL-40, HOM-HD-397, HOM-RCC-1.14, NY-ESO-1, NY-LU-12, NY-CO-13 and MAGE genes, were expressed in these malignancies, demonstrating that several human tumour types express multiple antigens capable of eliciting an immune response in the autologous host. This represents an alternative and more efficient approach to identify tumour markers, and offers distinct advantages over previously used techniques:
1) the use of fresh tumour specimens to produce the cDNA libraries obviates the need to culture tumour cells in vitro and therefore circumvents artefacts, such as loss or neo-antigen expression and genetic and phenotypic diversity generated by extended culture;
2) the analysis is restricted to antigen-encoding genes expressed by the tumour in vivo;
3) using cDNA expression cloning, the serological analysis (in contrast to autologous typing) is not restricted to cell surface antigens, but covers a more extensive repertoire of cancer-associated proteins (cytosolic, nuclear, membrane, etc.);
4) in contrast to techniques using monoclonal antibodies, SEREX uses poly-specific sera to scrutinise single antigens that are highly enriched in lytic bacterial plaques allowing the efficient molecular identification of antigens following sequencing of the cDNA. Subsequently the tissue-expression spectrum of the antigen can be determined by the analysis of the mRNA expression patterns using, for example, northern blotting and reverse transcription-PCR (RT-PCR), on fresh normal and malignant (autologous and allogeneic) tissues. Likewise, the prevalence of antibody in cohorts of cancer patients and normal controls can be determined.
   TACC1-D Identification

cDNA clone Ga55 encoding TACC1 was isolated from gastric cancer cDNA expression library by immunoscreening with autologous patient's serum using SEREX. This clone reacted exclusively with the patient's serum but not with sera from healthy individuals (n=35). The reactivity of autologous serum to TACC1 protein was also confirmed by Western blot analysis using a recombinantly expressed TACC1 fragment. Comparison of Ga55 cDNA (GenBank Accession number AY039239) with the previously published TACC1 sequence (AF049910) showed that Ga55 represents a TACC1 splice variant generated by inclusion of alternative 36-bp exon and that the clone contains a partial cDNA sequence truncated at both 5′ and 3′ ends. Additionally, alignment of corresponding ESTs indicated that several other 5′ variants of the transcript may be generated by alternative splicing. In order to analyse the exon composition of TACC1 mRNA 5′ variants expressed in gastric cancer tissue and to determine the transcription start sites of these mRNAs the inventors performed RNA-Ligase-Mediated Rapid Amplification of cDNA Ends (RLM-RACE) using a FirstChoise™ RLM-RACE kit (Ambion) according to manufacturer's protocol.

10 μg of total RNA was isolated from gastric cancer tissues and treated with Calf Intestinal Phosphatase to remove 5′-phosphates from un-capped RNAs, then cap structure was removed from full-length mRNA by Tobacco Acid Pyrophosphatase (TAP) and RNA adapters were ligated to mRNA molecules containing 5′phosphate. A random-primed reverse transcription and nested PCR with gene-specific and adapter-specific primers were performed.

TACC1-D
forward primer 5′-ccaagttctgcgccatggg-3′
reverse primer 5′-aatttcacttgttcagtagtc-3′
AD034
forward primer 5′-cttatctctcaaaggccatgg-3′
reverse primer 5′-gattttctaggagtgcaggg-3′

The RNA sample, which has not been treated with TAP, was carried through the adapter ligation and RT-PCR, as a negative control to demonstrate that the RLM-RACE products are generated by amplification of the 5′ ends of full-length (decapped) RNA. Two bands of approximately 240-bp and 280-bp were detected when gene-specific primers located in exon 4 were used. These PCR products were cloned using InsT/Aclone™PCR Product Cloning Kit (Fermentas, Lithuania) and at least 10 plasmid clones containing each PCR product were sequenced on AB1 PRISM 310 automatic sequencer (Applied Biosystems). Comparison of the obtained sequences to the published TACC1 mRNA sequence (here designated as TACC1-A) and to the working draft of human genome (www.ncbi.nln.nih.gov) showed that these RLM-RACE products represent three novel TACC1 mRNA variants, designated TACC1-B, TACC1-C and TACC1-D (FIG. 1B). The first exons of these transcripts were not present in the published TACC1-A mRNA, but comparison with the genomic sequence (NT_{—}008251) showed that exon 1a is located 53.65 Kb and exon 1b-82.3 Kb upstream from the first exon of TACC1-A, suggesting that these transcript variants are under the control of different promoters. The transcription start site in exon 1b seems to be fixed as no differences among individual clones were detected. In contrast, the start site in exon 1a is scattered within 100-bp region. No transcript variants corresponding to the clone Ga55 and published TACC1-A sequence were detected in RLM-RACE analyses likely reflecting an advantage for more abundant and/or shorter mRNA species in this PCR-based technique.

The inventors then designed a set of isoform-specific primers to analyse the expression of TACC1 isoforms in normal and cancerous tissues. The sequences of the primers are shown in Table 1 and their location is indicated in FIG. 1B.

TABLE 1

Primers used for amplification of TACC1
transcript variants and controls

| | | | | Size of |
|---|---|---|---|---|
| Isoform/ | | | No. of | product |
| gene | | Primer sequences (5′-3′) | cycles | (bp) |
| TACC1-A | F | AGGAGGAGGATTCGCAAGC | 35 | 387 |
| | R | TTGTTCCGAGGACTGCCGAG | | |
| TACC1-B | F | CCTCGCCGAAGAGGAGTGG | 37 | 252 |
| | R | TGGTAGACACAGGAACATTGG | | |
| TACC1-C | F | CCACGGAGACCGCGAGTG | 36 | 252 |
| | R | TGGTAGACACAGGAACATTGG | | |
| TACC1-D | F | CCAAGTTCTGCGCCATGGG | 38 | 112 |
| | R | AATTTCACTTGTTCAGTAGTC | | |
| TACC1-E | F | GAGAGATGCGAAATCAGCG | 35 | 432 |
| | R | TTGTTCCGAGGACTGCCGAG | | |
| TACC1-F | F | CTTTGACGAATCCATGGATCC | 31 | 129 |
| | R | AATTTCACTTGTTCAGTAGTC | | |
| TACC-CCD | F | AAATACGAAGAGACCCGGC | 28 | 349 |
| | R | TGTCCAGTTTCTCTTCTGCG | | |
| GAPDH | F | GTCATCCCTGAGCTAGACGG | 25 | 356 |
| | R | GGGTCTTACTCCTTGGAGGC | | |

Location of primers is indicated by arrows in FIG. 1B. TACC-CCD—region of TACC encoding coiled-coil domain, F—forward primer, R—reverse primer.

Initially when the primers used for amplification were located within exons 1b and 5, a 1500-bp band was detected in addition to the expected 318-bp (TACC1-B) product. Direct sequencing of this RT-PCR product revealed one more TACC1 splice variant (designated TACC1-E), however the complete 5′ end sequence for this variant is not known. The mRNA expression of the isoforms was analysed in a panel of normal tissues (brain, liver, heart, kidney, lung, trachea, (Clontech) spleen, colon, stomach, testis and ovary (Ambion)) and tumour and adjacent tissues of 10 patients diagnosed with gastric adenocarcinoma. Fragments of GAPDH and TACC1 coiled-coil domain (exons 8-11) were amplified as controls to demonstrate that equal amounts of total mRNA are used for analysis. Optimal cycling conditions (input cDNA and number of cycles) for the controls were determined so that the amount of PCR product is in liner relationship from the amount of input cDNA. Linearity of the amplification was confirmed by a series of PCR with 1.5-fold dilutions of input cDNA. In analysis of the isoform expression, additional cycles of amplification were performed to increase the sensitivity of the assay, which may reduce the linearity of amplification in some cases. Transcript variants A, B, C and E were expressed in all normal tissues analysed and no significant differences between cancerous and adjacent tissues were observed. From the normal tissues analysed, TACC1-F was strongly expressed only in brain and weakly detectable in lung and colon. TACC1-D was almost undetectable in any of normal tissues with only trace amounts detected in kidney and colon after 38 cycles of amplification. At the same cycling conditions relatively strong TACC1-D expression was observed in 5 out of 10 specimens of gastric cancer tissues while very faint signals were detected in two of the adjacent tissue samples. TACC1-F expression was detected in normal brain tissue and at a similar level in 6 specimens of gastric cancer, however it also was detectable as a weak signal in most adjacent tissues. Analysis of differentially expressed isoforms and controls is shown in FIG. 2B. The number of cycles required to yield a detectable product (shown in Table 1) is unlikely to represent the relative abundance of the isoforms due to variations in efficiency of the primers, therefore the inventors cannot estimate ratio of the isoforms. Co-amplification of TACC1-A/E and F, and TACC1-C/D showed that both TACC1-F and D are less abundant in gastric cancer cells than TACC1-A/E and C, respectively (FIG. 2C). Despite the overexpression TACC1-D and F variants in tumours the inventors did not observe significant differences in total TACC1 level (TACC-CCD) between cancerous and non-cancerous tissues of these patients. This shows that regulation of mRNA splicing rather than expression level of TACC1 is altered in gastric tumours. Both TACC1-F and D contain exon 4a that is not included in any other transcript variant. Presumably the splice sites of the alternative exon are “weaker” and are not recognised by the splicing machinery in normal tissues except brain. The mechanism of altered splice site selection in cancer cells is not known, although it has been shown that mutations or sequence polymorphisms in splice regulatory sequences, changes in splicing factors and activation of particular signal transduction pathways may modulate the use of alternative splice sites (Philips A. V., et al., Cell Mol. Life Sci., Vol. 57, pages 235-249). Alterations in the splicing pattern or efficiency of several genes have been implicated in tumour progression (for example, CD44, WT-1, C-CAM1) and susceptibility to cancer (for example, BRCA1, CYP3A).

Like mutations, altered splicing can serve as one of the mechanisms for the generation of protein diversity contributing to the selection of more aggressive tumour cells (Philips A. V., Supra and Cooper T. A., Am. J. Hum. Gend. (1997), Vol. 61, pages 259-266). Here the inventors show that the regulation of alternative splicing of TACC1 is perturbed in primary gastric tumours. Both of the differentially expressed isoforms can be exploited as biomarkers for gastric cancer and the study of their prognostic significance is currently being investigated. Although the function of the TACC1 isoforms is not known, the inventors propose that aberrant expression of TACC1-D and F isoforms appears to contribute to centrosome malfunction. Various centrosome abnormalities, including atypical size, shape and increased number, are observed in most of the common human cancers but little is known about the underlying genetic alterations. Centrosome defects are known to lead to the formation of multipolar spindles and chromosome segregation errors, see, for example, Salisbury J. L., J. Mamm. Gland. Biol. Neoplasia (2001), Vol. 6, pages 203-212; Sato N., et al, Cancer Genet. Cytogenet. (2002), Vol. 126, pages 13-19; Duensing SCX Munger K., Biochem. Bioplys. Acta. (2001), Vol. 1471, M81-M88; Marx J., Science (2001), Vol 292, pages 426-429.

The identified TACC1 isoforms differ in their N-terminal regions but share identical coiled-coil domain. The coiled-coil domain interacts with microtubules by cooperating with another microtubule-associated protein (Msps in Drosophila) which stabilises centrosomal microtubules (Lee, et al., Supra). TACC1-A protein is distributed in the cytoplasm and nucleus in interphase but it concentrates at centrosomes and on microtubules during mitosis; the N-terminal domain appear to be required for proper subcellular distribution during the cell cycle. In fact, TACC1-A and TACC1-E contains two nuclear localisation signals which are absent in the four shortest splice variants.

Experiments with Drosophila have shown that decreasing the level of D-TACC protein leads to the formation of abnormally short centrosomal microtubules and subsequently to severe mitotic defects (Gergely, et al., Supra). In contrast, overexpression of TACC-D leads to the formation of large, highly ordered protein aggregates around the centrosomes and an increase in the number and/or length of centrosomal microtubules (Lee, et al., Supra). When coiled-coil domains of human TACC proteins are overexpressed in HeLa cells, they form similar polymeric structures in the cytoplasm, full-length TACC1-A also forms polymers, but they are less compacted and clustered around the nucleus (Gergely, et al., Supra). This shows that perturbations in TACC gene expression could contribute to the mitotic defects and genetic instability. The inventors propose that deregulation of alternative splicing resulting in inappropriate expression of TACC1 isoforms in gastric cancer could result in the dysfunction of TACC1. It is possible that the formation of such protein aggregates might have served as an immunogenic stimuli in the cancer patients resulting in the production of anti-TACC1 antibodies. In the study, the antibody response to TACC1 was restricted to the autologous patient but interestingly, both TACC1 and 2 have been detected by SEREX in gastric cancer by Y. Obata (SEREX database). If the B-cell response to TACC1 in patients is elicited by the formation of protein aggregates as a consequence of deregulated TACC1 expression, given the restricted expression of, e.g. TACC1-D, some of the isoforms are a target for vaccine based immunotherapy.

Furthermore, the functional differences of the isoforms are likely to differ, thus making them a target for compounds affecting their activity.

TACC1-D is especially of interest because of its specific expression in relatively high amounts in gastrointestinal cancers.

AD034 Isolation

Tissue Specimens and Patient Sera

Colorectal cancer tissue and the adjacent non-cancerous tissue specimens from 15 patients undergoing surgery at the Latvian Oncology Center were resected and frozen in liquid nitrogen immediately after the surgery. Clinico-pathologic data, including histology, depth of invasion, lymph node and liver metastasis, Dukes' stage, etc., were obtained from the clinical records. In addition, serum samples were obtained from colon, stomach and breast cancer patients undergoing diagnostic procedures and from healthy volunteers. The study was approved by Committee of Medical Ethics of Latvia and the tissue samples and sera were collected after the patients' informed consent was obtained.

Isolation of Total RNA and Construction of cDNA Library

Total RNA was isolated from tumour and normal tissue samples, using Trizol reagent according to manufacturer's protocol (Life Technologies, Inc.). A cDNA expression library was constructed from tumour specimen of a moderately differentiated adenocarcinoma of colon. Poly(A)⁺ RNA was purified from total RNA using Dynabeads mRNA Purification kit (Dynal AS, Norway) and cDNA was ligated into the lambda Uni-SAP XR vector using Gigapack III Gold cloning kit (Stratagene GmbH). After in vitro packaging, a library containing 10⁶ primary cDNA clones was obtained and amplified once prior to immunoscreening.

Immunoscreening

Immunoscreening of the cDNA library was performed as described by Sahin, et al., (1995) Supra. Briefly, E. coli XL1 blue MRF′ cells were transfected with the recombinant phages, plated at a density of approx. 5000 pfu/150-mm plate (NZCYM-IPTG-agar) and following 8 hr. incubation at 37° C. transferred to nitrocellulose filters. In order to eliminate cDNA clones encoding human imunoglobins, filters were pre-screened with AP-conjugated rabbit anti-human secondary antibody (Pierce, USA) prior to incubation with sera, and reactive plaques were detected with 5-bromo-4chloro-3-indolyl-phosphate (BCIP)/nitroblue tetrazolium (NBT) and marked. Then filters were incubated with 1:250 diluted patient's serum, which had been previously preabsorbed with E. coli-phage lysate, serum-reactive clones were detected with AP-conjugated secondary antibody and visualised by incubating with BCIP/NBT. The reactive phage clones were subcloned to monclonality and converted to pBluescript phagemids. To assess frequencies of antibody responses to the SEREX-defined antigens in allogeneic sera, E. coli were transfected directly on the gridded agar plate, by spotting 1 μl of monoclonal positive phage (20-30 pfu/μl) side by side with non-recombinant phages. “Phage arrays” were screened with 1:200 diluted allogeneic sera as described above, excluding the IgG pre-screening step.

DNA Sequencing and Sequence Analysis

Phagemid DNA was purified using QIAprep Spin Miniprep kit (QIAGEN GmbH), analysed by EcoRI/XhoI restriction enzyme digestion and clones representing different cDNA inserts were sequenced using BigDye Terminator Cycle Sequencing Ready Reaction kit on an ABI PRISM 3100 genetic analyser (Applied Biosystems). Gene-specific primers were designated to obtain full insert sequences. Genes were identified by homology search through the GenBank data base (www.ncbi.nlm.nih.gov/BLAST). Chromosomal localisation and exon-intron organisation of the cDNAs was determined by comparison to the working draft of the human genome. Putative protein domains were predicted by scanning the sequences against PROSITE (www.expasy.org) and by using tools for sequence analysis at the SEREX web-site (www-ludwig.unil.ch/SEREX).

Western Blot Analysis

Immunoreactivity to the recombinant proteins in serum-reactive clones was confirmed by Western blot analysis. E. coli XL1-Blue cells were transformed with the recombinant pBluescript phagemids excised from the Uni-ZAP XR vector. The cells were grown in LB medium with ampicillin to OD of 0.4 at 540 nm and then transcription from the lacZ promoter was induced with 2 mM IPTG. Samples of the bacterial cultures were collected before induction and 3 and 5 after the protein expression was induced. The cells were lysed with 3×Laemli buffer, lysates were separated by SDS-PAGE and blotted to Hybond c-extra filters (Amersham Biosciences). The filters were blocked with fat-free milk, incubated with the autologous patient serum and antigen-antibody complexes were detected with HRP-conjugated rabbit anti-human antibody using an ECL detection system (Amersham Biosciences).

Comparative RT-PCR Analysis

The mRNA expression pattern of SEREX-defined antigens was analysed by RT-PCR using a panel of normal tissue RNA (whole brain, liver, heart, kidney, lung, trachea) (Clontech), (stomach, colon, spleen, testis, ovary) (Ambion), PBLs and a specimen of colon cancer of the autologous patient. Relative mRNA levels were compared between cancerous and adjacent non-cancerous tissues of 15 patients by comparative RT-PCR. The first-strand cDNA was synthesised from 4 μg of total RNA primed with oligo-dT(18) and random hexamer primers using a First-Strand cDNA Synthesis Kit (Fermentas, Lithuania). Gene specific PCR primers located within different exons were designed to amplify cDNA fragments (250-350 bp in length) of AD034 genes and GAPDH and β-actin as internal standard genes. One fiftieth of RT mixture was amplified in GeneAmp PCR System 2400 thermal cycler (Perkin-Elmer Corp.) in a total reaction volume of 20 μl containing 10 pmole of each primer, 200 μM dNTPs and 2 U of Taq polymerase (Fermentas, Lithuania). Optimisation of cycling conditions (amount of input cDNA and number of cycles) was performed as described by Toh, et al., Int. J. Cancer (1997), Vol. 72, page 459. Amplification of all target genes was performed simultaneously, at the same cycling conditions (45 s at 94° C., 30 s at 58° C., 45 s at 72° C.), except for the number of cycles that was different for the amplification of each target gene. The primer sequences, number of cycles used and length of PCR products are shown in Table 2. The quantity of RT-PCR products was determined densitomertrically after scanning the ethidium bromide stained gel on digital gel documentation and analysis system GDS8000 (Ultra-Violet Products Ltd., UK) and the intensities of bands were calculated using GelWorks software. Standard curves of amplification of each target gene were constructed from a series of PCRs with ten 1.5-fold dilutions of the colon cancer cDNA. Amounts of PCR products were linearly dependent from input cDNA over 10-fold dilutions of cDNA. The relative amounts of target mRNAs were normalised to GAPDH and β-actin. The obtained values in tumours (T) were compared to those in matched normal epithelium (N) and T/N ratios were calculated for each mRNA in each patient's tissue samples. Each reaction was performed in duplicate.

5′ RLM-RACE of Co23 (AD034)

The full-length 5′ end of Co23 cDNA sequence was cloned from colon cancer tissues of autologous patient using FirstChoise™ RLM-RACE kit (Ambion) according to the manufacturer's protocol. Briefly, 10 μg of total RNA were treated with Calf Intestinal Phosphatase to remove 5′-phosphates from uncapped RNAs (degraded mRNA, rRNA, tRNA or DNA), then the cap structure was removed from the full length mRNA by Tobacco Acid Pyrophosphatase and RNA adapters were ligated to mRNA molecules containing 5′phosphate. A random-primed RT-nested PCR with gene-specific and adapter-specific primers was performed, products were cloned using InsT/Aclone™PCR Product Cloning Kit (Fermentas, Lithuania) and multiple clones were sequenced.

TABLE 2

Primers used for expression analysis of
SEREX-defined antigens

| | | | | Size of |
|---|---|---|---|---|
| | | | No. of | product |
| Gene | | Primer sequences (5′-3′) | cycles | (bp) |
| AD034 | F | CTTATCTCTCAAAGGCCATGG | 28 | 276 |
| | R | GATTTTCTAGGAGTGCAGGG | | |
| AD034^{b} | F | ATGATGATGACTGGGACTGG | 32 | 176v144 |
| (ex.2-3) | R | GTAAGACCTTGTCTGCTGG | | |
| β-actin | F | AGTGTGACGTGGACATCCG | 20 | 351 |
| | R | AATCTCATCTTGTTTTCTGCGC | | |
| GAPDH | F | GTCATCCCTGAGCTAGACGG | 25 | 356 |
| | R | GGGTCTTACTCCTTGGAGGC | | |

^{b}These sets of primers were used for analysis of expression of RHAMM and AD034 splice variants, respectively.
F-forward primer, R-reverse primer.

Results

Immunoscreening and Identification of Immunoreactive cDNA Clones

Fourteen serum-reactive cDNA clones were detected by immunoscreening of 8×19⁵ pfu from a colon cancer cDNA expression library with autologous patient's serum. The clones were purified, full-length sequences of their cDNA inserts were obtained and the genes were identified by homology search through the GenBank data base.

mRNA Expression of SEREX-Defined Antigens

mRNA expression of SEREX-defined antigens was analysed by RT-PCR in normal tissues (brain, liver, heart, kidney, lung, trachea, spleen, colon, stomach, testis, ovary and PBLs) and in a specimen of colon cancer tissue of the autologous patient. Cycling conditions and the optimal number of cycles were chosen so that the PCR products were at the liner phase of amplification. GAPDH and β-actin were used as controls for RNA integrity and quantity. This allows to assess the abundance of each mRNA in normal tissues relative to the autologous colon cancer tissue. Co23 was expressed in testis, spleen, colon, stomach and colon cancer tissues (FIG. 2).

Comparison of mRNA Levels in Colon Cancer and Adjacent Non-Cancerous Tissues

To determine whether the antigens showing relatively high expression in the autologous tumour are overexpressed in other colorectal cancer, the inventors compared their relative mRNA levels between cancerous and paired adjacent tissue specimens of 15 patients with colorectal cancer by RT-PCR. The conditions for amplification of each target gene were optimised so that the amount of PCR product was in liner relationship to the amount of input cDNA, at least over 10-fold dilution of input cDNA. GAPDH and β-actin were used as internal controls. An example of analysis is shown in FIG. 3. Relative quantities of RT-PCR products were determined by densitometric analysis, the amounts of target cDNAs were normalised to that of β-actin or GAPDH and tumour/normal ratios were calculated. Ratios≧2 (the mean values of two independent experiments) were considered to represent significant overexpression. They observed a 2.0-4.8-fold increase of Co23 (AD034) in 4 specimens of colon cancer when compared to the adjacent tissues (normalised to β-actin).

Cloning of AD034 mRNA 5′ Variants

Clone Co23 contains a partial cDNA sequence encoding hypothetical protein AD034. The longest ORF encodes a 561-amino acid protein of approximately 64.6 kDa. Comparison of the predicted amino acid sequence with PROSITE and Pfam databases revealed a similarity to the RIO1/ZK632.3/MJ0444 protein family (aa 186-380), the tyrosine kinase active-site signature (aa 313-325) and the aspartic acid and lysine-rich regions (aa 10-66 and 514-561, respectively). To determine the transcription start site and to search for possible sequence variations in the 5′ region of AD034 that was absent in clone Co23, 5′RLM-RACE analysis was performed using total RNA from tumour tissues of the autologous patient 5′ ends of the sequenced RLM-RACE clones differed by 154-bp, indicating that the transcription start site of AD034 is scattered within this region. The longest RACE clone extended the AD034 mRNA sequence by 37-bp, however no additional translation initiation site was found. Of the 8 clones sequenced, three contained an insertion of 32-bp (submitted to GenBank, AY094356). Alignment with the genomic sequence (NT_{—}023412) showed that the inserted 32-bp are derived from the intronic sequence flanking exon 3 and presumably are included in the mRNA by use of cryptic splice site. The insertion shifts the reading frame and introduces a stop codon resulting in a truncated ORF of 91aa. RT-PCR analyses of expression of the splice variants showed that the transcript containing the 32-bp sequence is just a minor mRNA variant and is detectable in all normal tissues where AD034 is expressed and no significant differences in the ratios of either of the splice variants were observed between colorectal tumours and adjacent normal tissues.

### Discussion

Clone Co23 encodes a hypothetical protein AD034. Analysis of the predicted amino acid sequence revealed a tyrosine kinase motif and a similarity to RIO1/ZK632.3/MJ0444 family—evolutionary related uncharacterised proteins. The inventors observed a relative upregulation of AD034 mRNA expression in several colon cancer cases, however the significance of AD034 expression in cancer development is unknown. The inventors also cloned a novel AD034 transcript variant, generated by use of a cryptic splice site. Translation of this transcript results in a truncated protein of 91 amino acids. However, RT-PCR analysis showed that the novel transcript variant represents less than 10% of the AD034 mRNA and is also detectable in several normal adult tissues, including normal colon thus showing that expression of this splice variant is not likely to be associated with immune recognition of AD034 in cancer patients, however the biological role of this isoform remains to be investigated.

1. A method of detecting or monitoring cancer comprising the step of detecting or monitoring elevated levels of (a) a nucleic acid molecule comprising a sequence selected from the group consisting of SEQ ID NO: 1, SEQ ID NO: 2, and SEQ ID NO: 3 in a sample from a patient or (b) a protein or peptide comprising an amino acid sequence encoded by a nucleic acid sequence selected from the group consisting of SEQ ID NO: 1, SEQ ID NO: 2 and SEQ ID NO: 3.

2. A method according to claim 1 wherein an isolated nucleic acid molecule comprising a sequence selected from the group consisting of SEQ ID NO: 1, SEQ ID NO: 2 and SEQ ID NO: 3 is used in the step of detecting or monitoring.

3. A method according to claim 1 wherein a nucleic acid probe which is capable of hybridising under high stringency conditions to an isolated nucleic acid molecule comprising a sequence selected from the group consisting of SEQ ID NO: 1, SEQ ID NO: 2 and SEQ ID NO: 3 is used in the step of detecting or monitoring.

4. A method of detecting or monitoring cancer according to claim 1 wherein a nucleic acid molecule or probe comprising a sequence selected from the group consisting of SEQ ID NO: 1, SEQ ID NO: 2 and SEQ ID NO: 3 is used in combination with a reverse transcription polymerase chain reaction (RT-PCR).

5. A method of detecting or monitoring cancer according to claim 1 wherein in the step of detecting or monitoring employs a nucleic acid molecule or probe which is capable of hybridising under high stringency conditions to an isolated nucleic acid molecule comprising a sequence selected from the group consisting of SEQ ID NO: 1, SEQ ID NO: 2 and SEQ ID NO: 3 in combination with a reverse transcription polymerase chain reaction (RT-PCR).

6. A method according to claim 1 comprising the use of an antibody selective for said protein or peptide to detect the protein or peptide.

7. A method according to claim 6 wherein an Enzyme-linked Immunosorbant Assay (ELISA) is used to detect the protein or peptide.

8. A method according to claim 1, wherein the cancer is a gastro-intestinal cancer.

9. A kit comprising
(a) an isolated nucleic acid molecule comprising a sequence selected from the group consisting of SEQ ID NO: 1, SEQ ID NO: 2 and SEQ ID NO: 3;
(b) an antibody selective for a protein or peptide comprising an amino acid sequence encoded by a nucleic acid sequence selected from the group consisting of SEQ ID NO: 1, SEQ ID NO: 2, and SEQ ID NO: 3 or
a nucleic acid probe which is capable of hybridising under high stringency conditions to an isolated nucleic acid molecule comprising a sequence selected from the group consisting of SEQ ID NO: 1, SEQ ID NO: 2 and SEQ ID NO: 3.

10. A method of prophylaxis or treatment of cancer comprising administering to a patient a pharmaceutically effective amount of
(a) nucleic acid molecule comprising a nucleic acid sequence selected from the group consisting of SEQ ID NO: 1, SEQ ID NO: 2, SEQ ID NO: 3, and pharmaceutically effective fragments thereof,
(b) a nucleic acid molecule hybridisable under high stringency conditions to a nucleic acid molecule comprising a nucleic acid sequence selected from the group consisting of SEQ ID NO: 1, SEQ ID NO: 2, SEQ ID NO: 3, and pharmaceutically effective fragments thereof,
(c) a protein or peptide comprising an amino acid sequence encoded by a nucleic acid sequence selected from the group consisting of SEQ ID NO: 1, SEQ ID NO: 2, SEQ ID NO: 3, and pharmaceutically effective fragments thereof, or
(d) of an antibody capable of specifically binding a protein or peptide comprising an amino acid sequence encoded by a nucleic acid sequence selected from the group consisting of SEQ ID NO: 1, SEQ ID NO: 2 and SEQ ID NO: 3.

11. A method according to claim 10, wherein the cancer is a gastro-intestinal cancer.

12. A vaccine comprising
(a) a nucleic acid molecule having a nucleic acid sequence selected from the group consisting of SEQ ID NO: 1, SEQ ID NO: 2, SEQ ID NO: 3, and pharmaceutically effective fragments thereof; and a pharmaceutically acceptable carrier, or
(b) a protein or peptide comprising an amino acid sequence encoded by a nucleic acid sequence selected from the group consisting of SEQ ID NO: 1, SEQ ID NO: 2, SEQ ID NO: 3, and pharmaceutically effective fragments thereof; and a pharmaceutically acceptable carrier.

13. An isolated mammalian nucleic acid molecule which codes for the following amino acid sequence:
MSRVVPGQFDDADSSDSENRDLKTVKEKDDILFEDLQDNVNENGEGEIED
EEEEGYDDDDDDWDWDEGVGKLAKGYVWNGGSNPQANRQTSDSSSAKMST
PADKVLRKFENKINLDKLNVTDSVINKVTEKSRQKEADMYRIKDKADRAT
VEQVLDPRTRMILFKMLTRGIITEINGCISTGKEANVYHASTANGESRAI
KIYKTSILVFKDRKYVSGEFRFRHGYCKGNPRKMVKTWAEKEMRNLIRLN
TAEIPCPEPIMLRSHVLVMSFIGKDDMPAPLLKNVQLSESKARELYLQVI
QYMRRMYQDARLVHADLSEFNMLYHGGGVYVSQSVEHDHPHALEFLRKDC
ANVNDFFMRHSVAVMTVRELFEFVTDPSITKENMDAYLSKAMEIASQRTK
EERSSQDHVDEEVFKRAYIPRTLNEVKNYERDMDIIMKLKEEDMAMNAQQ
DNILYQTVTGLKKDLSGVQKVPALLENQVEERTCSDSEDIGSSECDDTDS
EDHARPKKHTTDPDIDKKERKKMVKEAQREKRKNKIPKHVKKRKEKTAKT
KKGK
or a variant or a fragment thereof which encodes a prostate-associated antigen which is expressed in higher than normal concentrations in prostate cancer cells.

14. A vector comprising an isolated mammalian nucleic acid molecule according to claim 13.

15. A nucleic acid molecule comprising at least 15 nucleotides, the nucleic acid molecule being capable of hybridising to a molecule according to claim 13 under high stringency conditions.

16. An isolated protein or peptide comprising an amino acid sequence obtainable from a nucleic acid molecule according to claim 13.

17. An isolated protein or peptide comprising an amino acid sequence obtainable from a nucleic acid molecule according to claim 14.

18. An isolated protein or peptide comprising an amino acid sequence obtainable from a nucleic acid molecule according to claim 15.

19-20. (canceled)


SEQUENCE LISTING (26 sequences)

SEQ ID NO: 1 - DNA, 4340 bp, Homo sapiens
acagccgcccgccgcccagcacaggagggtgcagccccggccccaagttctgcgccatgggaggctcccactctcagaccccgaggggccgggaacccgccggggagaggcacccgagacccacggagaccgcgactactgaacaagtgaaatttctctgttttctgttgagtggctgtaaggtgaagaagcatgaaactcagtctctcgccctggatgcatgttctcgggatgaaggggcagtgatctcccagatttcagacatttctaatagggatggccatgctactgatgaggagaaactggcatccacgtcatgtggtcagaaatcagctggtgccgaggtgaaaggtgagccagaggaagacctggagtactttgaatgttccaatgttcctgtgtctaccataaatcatgcgttttcatcctcagaagcaggcatagagaaggagacgtgccagaagatggaagaagacgggtccactgtgcttgggctgctggagtcctctgcagagaaggcccctgtgtcggtgtcctgtggaggtgagagccccctggatgggatctgcctcagcgaatcagacaagacagccgtgctcaccttaataagagaagagataattactaaagagattgaagcaaatgaatggaagaagaaatacgaagagacccggcaagaagttttggagatgaggaaaattgtagctgaatatgaaaagactattgctcaaatgattgaagatgaacaaaggacaagtatgacctctcagaagagcttccagcaactgaccatggagaaggaacaggccctggctgaccttaactctgtggaaaggtccctttctgatctcttcaggagatatgagaacctgaaaggtgttctggaagggttcaagaagaatgaagaagccttgaagaaatgtgctcaggattacttatccagagttaaacaagaggagcagcgataccaggccctgaaaatccacgcagaagagaaactggacaaagccaatgaagagattgctcaggttcgaacaaaagcaaaggctgagagtgcagctctccatgctggactccgcaaagagcagatgaaggtggagtccctggaaagggccctgcagcagaagaaccaagaaattgaagaactgacaaaaatctgtgatgagctgattgcaaagctgggaaagactgactgagacactccccctgttagctcaacagatctgcatttggctgcttctcttgtgaccacaattatcttgccttatccaggaataattgcccctttgcagagaaaaaaaaaaacttaaaaaaagcacatgcctactgctgcctgtcccgctttgctgccaatgcaacagccctggaagaaaccctagagggttgcatagtctagaaaggagtgtgacctgacagtgctggagcctcctagtttccccctatgaaggttcccttaggctgctgagtttgggtttgtgatttatctttagtttgttttaaagtcatctttactttcccaaatgtgttaaatttgtaactcctctttggggtcttctccaccacctgtctgatttttttgtgatctgtttaatcttttaattttttagtatcagtggttttatttaaggagacagtttggcctattgttacttccaatttataatcaagaaggggctctggatccccttttaaattacacacactctcacacacatacatgtatgtttatagatgctgctgctcttttccctgaagcatagtcaagtaagaactgctctacagaaggacatatttccttggatgtgagaccctattttgaaatagagtcctgactcagaacaccaacttaagaatttgggggattaaagatgtgaagaccacagtcttgggttttcatatctggagaagactatttgccatgacgttttgttgccctggtatttggacactcctcagctttaatgggtgtggcccctttagggttagtcctcagactaatgatagtgtctgctttctgcatgaacggcaatatgggactccctccaagctagggtttggcaagtctgccctagagtcatttactctcctctgcctccatttgttaatacagaatcaacatttagtcttcattatctttttttttttttttgagacagagtttcgatctattttaagtatgtgaagaaaatctacttgtaaaaggctcagatcttaattaaaaggtaattgtagcacattaccaattataaggtgaagaaatgtttttttcccaagtgtgatgcattgttcttcagatgttgaaaagaaagcaaaaaataccttctaacttaagacagaatttttaacaaaatgagcagtaaaagtcacatgaaccactccaaaaatcagtgcattttgcatatttttaaacaaagacagcttgttgaatactgagaagaggagtgcaaggagaaggtctgtactaacaaagccaaattcctcaagctcttactggactcagttcagagtggtgggccattaaccccaacatggaatttttccatataaatctcaatgaattccctttcatttgaataggcaaacccaaatccatgcaagtgttttaaagcactgtcctgtcttaatcttacatgctgaaagtcttcatggtgatatgcactatattcagtatacgtatgttttcctacttctcttgtaaaactgttgcatgatccaacttcagcaatgaattgtgcctagtggagaacctctatagatcttaaaaaatgaattattctttagcagtgtattactcacatgggtgcaatctttagccccagggaggtcaataatgtcttttaaagccagaagtcacattttaccaatatgcatttatcataattggtgcttaggctgtatattcaagcctgttgtcttaacattttgtataaaaaagaacaacagaaattatctgtcatttgagaagtggcttgacaatcatttgagctttgaaagcagtcactgtggtgtaatatgaatgctgtcctagtggtcatagtaccaagggcacgtgtctccccttggtataactgatttcctttttagtcctctactgctaaataagttaattttgcattttgcagaaagaaacattgattgctaaatctttttgctgctgtgttttggtgttttcatgtttacttgttttatattgatctgttttaagtatgagaggcttatagtgccctccattgtaaatccatagtcatctttttaagcttattgtgtttaagaaagtagctatgtgttaaacagaggtgatggcagcccttccctagcacactggtggaagagaccccttaagaacctgaccccagtgaatgaagctgatgcacagggagcaccaaaggaccttcgttaagtgataattgtcctggcctctcagccatgaccgttatgaggaaatatcccccattcgaacttaacagatgcctcctctccaaagagaattaaaatcgtagcttgtacagatcaagagaatatactgggcagaatgaagtatgtttgtttatttttctttaaaaataaaggattttggaactctggagagtaagaatatagtatagagtttgcctcaacacatgtgagggccaaataacctgctagctaggcagtaataaactctgttacagaagagaaaaagggccgggcacagtggcttattcctgtaatcccaacactgtggaaggccgaggcaggaggatcacttgagtccaggagtttgaaacctacctaggcaacatggtgaaaccttgtctctaccaaaataaaaattagctgggcatggtggcacgtgcctgtggtcccagctacttgggaggctgaggtgggagcctgggaggtcaaggctgcagtgagccatgatcatgccactgcactccatcctgggtgacagcaagatcttgtctcaaaaaaaaaaaaaaaaaaaaaaaacccaggagtgaaaaaggaaagtaaaaggcagctgctggcctaaatgttggtttgggaatattaggtgatcctgttgaaattctggatccaaagcaatttctttagcttttgactttgccaaagtgtaaatagcctttatccaccagttttttaaggggggaatgcaacgggaggccaactgaacaattccccccgtggctgcccagatagtcacagtcaaggttggagagtctccttccagccagggacctacccaaaccttttgttctgtaaaactgctctggaaataccgggaagcccagttttctcacgtggtttctagcttcttcggactcagcccaatttaggagtgccgaagcacatgatgg

SEQ ID NO: 2 - DNA, 2495 bp, Homo sapiens
gggtggtggatctgtcggtcccgttttcccgtcgcacgtggtggccactgttggcttctgaatggtttgcaaggcggatatccacgccaaggcctttggatcggccgtgggtacatccgtctgagccgttcctttccatcgcagagcggcggcctccggcggcgctctccagtcatggactaccggcggcttctcatgagccgggtggtccccgggcaattcgacgacgcggactcctctgacagtgaaaacagagacttgaagacagtcaaagagaaggatgacattctgtttgaagaccttcaagacaatgtgaatgagaatggtgaaggtgaaatagaagatgaggaggaggagggttatgatgatgatgatgatgactgggactgggatgaaggagttggaaaactcgccaagggttatgtctggaatggaggaagcaacccacaggcaaatcgacagacctccgacagcagttcagccaaaatgtctactccagcagacaaggtcttacggaaatttgagaataaaattaatttagataagctaaatgttactgattccgtcataaataaagtcaccgaaaagtctagacaaaaggaagcagatatgtatcgcatcaaagataaggcagacagagcaactgtagaacaggtgttggatcccagaacaagaatgattttattcaagatgttgactagaggaatcataacagagataaatggctgcattagcacaggaaaagaagctaatgtataccatgctagcacagcaaatggagagagcagagcaatcaaaatttataaaacttctattttggtgttcaaagatcgggataaatatgtaagtggagaattcagatttcgtcatggctattgtaaaggaaaccctaggaaaatggtgaaaacttgggcagaaaaagaaatgaggaacttaatcaggctaaacacagcagagataccatgtccagaaccaataatgctaagaagtcatgttcttgtcatgagtttcatcggtaaagatgacatgcctgcaccactcttgaaaaatgtccagttatcagaatccaaggctcgggagttgtacctgcaggtcattcagtacatgagaagaatgtatcaggatgccagacttgtccatgcagatctcagtgaatttaacatgctgtaccacggtggaggcgtgtatatcattgacgtgtctcagtccgtggagcacgaccacccacatgccttggagttcttgagaaaggattgcgccaacgtcaatgatttctttatgaggcacagtgttgctgtcatgactgtgcgggagctctttgaatttgtcacagatccatccattacacatgagaacatggatgcttatctctcaaaggccatggaaatagcatctcaaaggaccaaggaagaacggtctagccaagatcatgtggatgaagaggtgtttaagcgagcatatattcctagaaccttgaatgaagtgaaaaattatgagagggatatggacataattatgaaattgaaggaagaggacatggccatgaatgcccaacaagataatattctataccagactgttacaggattgaagaaagatttgtcaggagttcagaaggtccctgcactcctagaaaatcaagtggaggaaaggacttgttctgattcagaagatattggaagctctgagtgctctgacacagactctgaagagcagggagaccatgcccgccccaagaaacacaccacggaccctgacattgataaaaaagaaagaaaaaagatggtcaaggaagcccagagagagaaaagaaaaaacaaaattcctaaacatgtgaaaaaaagaaaggagaagacagccaagacgaaaaaaggcaaatagaatgagaaccatattatgtacagtcattttcctcagttccttttctcgcctgaactcttaagctgcatctggaagatggcttattggttttaaccagattgtcatcgtggcactgtctgtgaagacggattcaaatgttttcatgtaactatgtaaaaagctctaagctctagagtctagatccagtcactgactctgtctggtgttgacagaggatttatttaagctattattttaataaagaactttgtacatttttatttttatatttttttctcttacaaatatgtttttggaagcatgataaatgtttaaatgtagtcaacatctgtaactcttacatgagtgtccagaggcactcatgggaaaattggttttgctttctttgtacacaccagagacccatctgaggtcatctgattataaggccatgtttatataaagggaatttcacccacagttcagctggctgttgattttcactgcaactctgcctttgtgtgtattggcgatcatttgtaatgctcttacacttcgtctttaatgttctttttggagttaggacctctcagttcataaagttttttacaattcaaaaaaaaaaaaaaaaaaaa

SEQ ID NO: 3 - DNA, 2527 bp, Homo sapiens
gggtggtggatctgtcggtcccgttttcccgtcgcacgtggtggccactgttggcttctgaatggtttgcaaggcggatatccacgccaaggcctttggatcggccgtgggtacatccgtctgagccgttcctttccatcgcagagcggcggcctccggcggcgctctccagtcatggactaccggcggcttctcatgagccgggtggtccccgggcaattcgacgacgcggactcctctgacagtgaaaacagagacttgaagacagtcaaagagaaggatgacattctgtttgaagaccttcaagacaatgtgaatgagaatggtgaaggtgaaatagaagatgaggaggaggagggttatgatgatgatgatgatgactgggactgggatgaaggagttggaaaactcgccaagggttatgtctggaatggaggaagcaacccacagctagtgccttagactctggaattcccttctaggcaaatcgacagacctccgacagcagttcagccaaaatgtctactccagcagacaaggtcttacggaaatttgagaataaaattaatttagataagctaaatgttactgattccgtcataaataaagtcaccgaaaagtctagacaaaaggaagcagatatgtatcgcatcaaagataaggcagacagagcaactgtagaacaggtgttggatcccagaacaagaatgattttattcaagatgttgactagaggaatcataacagagataaatggctgcattagcacaggaaaagaagctaatgtataccatgctagcacagcaaatggagagagcagagcaatcaaaatttataaaacttctattttggtgttcaaagatcgggataaatatgtaagtggagaattcagatttcgtcatggctattgtaaaggaaaccctaggaaaatggtgaaaacttgggcagaaaaagaaatgaggaacttaatcaggctaaacacagcagagataccatgtccagaaccaataatgctaagaagtcatgttcttgtcatgagtttcatcggtaaagatgacatgcctgcaccactcttgaaaaatgtccagttatcagaatccaaggctcgggagttgtacctgcaggtcattcagtacatgagaagaatgtatcaggatgccagacttgtccatgcagatctcagtgaatttaacatgctgtaccacggtggaggcgtgtatatcattgacgtgtctcagtccgtggagcacgaccacccacatgccttggagttcttgagaaaggattgcgccaacgtcaatgatttctttatgaggcacagtgttgctgtcatgactgtgcgggagctctttgaatttgtcacagatccatccattacacatgagaacatggatgcttatctctcaaaggccatggaaatagcatctcaaaggaccaaggaagaacggtctagccaagatcatgtggatgaagaggtgtttaagcgagcatatattcctagaaccttgaatgaagtgaaaaattatgagagggatatggacataattatgaaattgaaggaagaggacatggccatgaatgcccaacaagataatattctataccagactgttacaggattgaagaaagatttgtcaggagttcagaaggtccctgcactcctagaaaatcaagtggaggaaaggacttgttctgattcagaagatattggaagctctgagtgctctgacacagactctgaagagcagggagaccatgcccgccccaagaaacacaccacggaccctgacattgataaaaaagaaagaaaaaagatggtcaaggaagcccagagagagaaaagaaaaaacaaaattcctaaacatgtgaaaaaaagaaaggagaagacagccaagacgaaaaaaggcaaatagaatgagaaccatattatgtacagtcattttcctcagttccttttctcgcctgaactcttaagctgcatctggaagatggcttattggttttaaccagattgtcatcgtggcactgtctgtgaagacggattcaaatgttttcatgtaactatgtaaaaagctctaagctctagagtctagatccagtcactgactctgtctggtgttgacagaggatttatttaagctattattttaataaagaactttgtacatttttatttttatatttttttctcttacaaatatgtttttggaagcatgataaatgtttaaatgtagtcaacatctgtaactcttacatgagtgtccagaggcactcatgggaaaattggttttgctttctttgtacacaccagagacccatctgaggtcatctgattataaggccatgtttatataaagggaatttcacccacagttcagctggctgttgattttcactgcaactctgcctttgtgtgtattggcgatcatttgtaatgctcttacacttcgtctttaatgttctttttggagttaggacctctcagttcataaagttttttacaattcaaaaaaaaaaaaaaaaaaaa

SEQ ID NO: 4 - protein, 561 aa, Homo sapiens [sequence omitted]

SEQ ID NO: 5 - DNA, 19 bp, Artificial Sequence
  Oligonucloeotide primer TACC1-D Forwards
ccaagttctgcgccatggg

SEQ ID NO: 6 - DNA, 21 bp, Artificial Sequence
  Oligonucloeotide primerTACC1-D Reverse
aatttcacttgttcagtagtc

SEQ ID NO: 7 - DNA, 21 bp, Artificial Sequence
  Oligonucloeotide primer AD034 Forwards
cttatctctcaaaggccatgg

SEQ ID NO: 8 - DNA, 20 bp, Artificial Sequence
  Oligonucloeotide primer AD034 Reverse
gattttctaggagtgcaggg

SEQ ID NO: 9 - DNA, 19 bp, Artificial Sequence
  Oligonucloeotide primer TACC1-A Forwards
aggaggaggattcgcaagc

SEQ ID NO: 10 - DNA, 20 bp, Artificial Sequence
  Oligonucloeotide primer TACC1-A Reverse
ttgttccgaggactgccgag

SEQ ID NO: 11 - DNA, 19 bp, Artificial Sequence
  Oligonucloeotide primerTACC1-B Forwards
cctcgccgaagaggagtgg

SEQ ID NO: 12 - DNA, 21 bp, Artificial Sequence
  Oligonucloeotide primer TACC1-B Reverse
tggtagacacaggaacattgg

SEQ ID NO: 13 - DNA, 18 bp, Artificial Sequence
  Oligonucloeotide primerTACC1-C Forwards
ccacggagaccgcgagtg

SEQ ID NO: 14 - DNA, 21 bp, Artificial Sequence
  Oligonucloeotide primer TACC1-C Reverse
tggtagacacaggaacattgg

SEQ ID NO: 15 - DNA, 19 bp, Artificial Sequence
  Oligonucloeotide primer TACC1-E Forwards
gagagatgcgaaatcagcg

SEQ ID NO: 16 - DNA, 20 bp, Artificial Sequence
  Oligonucloeotide primer TACC1-E Reverse
ttgttccgaggactgccgag

SEQ ID NO: 17 - DNA, 21 bp, Artificial Sequence
  Oligonucloeotide primer TACC1-F Forwards
ctttgacgaatccatggatcc

SEQ ID NO: 18 - DNA, 21 bp, Artificial Sequence
  Oligonucloeotide primer TACC1-F Reverse
aatttcacttgttcagtagtc

SEQ ID NO: 19 - DNA, 19 bp, Artificial Sequence
  Oligonucloeotide primerTACC-CCD Forwards
aaatacgaagagacccggc

SEQ ID NO: 20 - DNA, 20 bp, Artificial Sequence
  Oligonucloeotide primer TACC-CCD Reverse
tgtccagtttctcttctgcg

SEQ ID NO: 21 - DNA, 20 bp, Artificial Sequence
  Oligonucloeotide primerGAPDH Forwards
gtcatccctgagctagacgg

SEQ ID NO: 22 - DNA, 20 bp, Artificial Sequence
  Oligonucloeotide primer GAPDH Reverse
gggtcttactccttggaggc

SEQ ID NO: 23 - DNA, 20 bp, Artificial Sequence
  Oligonucloeotide primer AD034b (ex2-3) Forwards
atgatgatgactgggactgg

SEQ ID NO: 24 - DNA, 19 bp, Artificial Sequence
  Oligonucloeotide primer AD034b (ex2-3) Reverse
gtaagaccttgtctgctgg

SEQ ID NO: 25 - DNA, 19 bp, Artificial Sequence
  Oligonucloeotide primer B-actin Forwards
agtgtgacgtggacatccg

SEQ ID NO: 26 - DNA, 22 bp, Artificial Sequence
  Oligonucloeotide primer B-actin Reverse
aatctcatcttgttttctgcgc